SEQUENCE LISTING (5 sequences)

SEQ ID NO: 1 - DNA, 389 bp, Bos sp.
catatgaaagaaacggctgcggcgaaatttgaacgccagcacatggatagcagcaccagcgcggcgagcagcagcaactactgtaaccagatgatgaaaagccgtaacttaaccaaagatcgttgtaaaccggtgaacacctttgtgcacgaaagcttagcggatgtgcaggcggtgtgcagccagaaaaacgtggcgtgtaaaaacggacagaccaactgctatcagagctacagcaccatgagcattaccgattgccgcgaaaccggtagcagcaaatatccgaactgtgcgtacaaaaccacccaggcgaacaaacatattattgtggcgtgtgaaggaaacccgtatgtgccggtgcattttgatgcgagcgtctaatagtcgac

SEQ ID NO: 2 - DNA, 389 bp, Bos sp.
catatgaaagaaacggctgcggcgaaatttgagcgccagcacatggacagctccaccagcgctgcctcgagctcgaattactgtaaccagatgatgaagtctcgtaacctgactaaagaccgttgtaagccggtgaacacgttcgtacacgaaagtttagcagatgtacaggccgtttgcagtcagaaaaatgtggcatgtaaaaacggacaaacgaattgctatcaaagttactctacaatgagcattaccgattgccgcgaaaccggttcctcaaaatatcctaattgtgcctacaaaaccactcaggcaaacaaacatattatcgtggcgtgcgagggcaacccgtatgtcccagttcactttgatgcgtcagtctaatagtcgac

SEQ ID NO: 3 - DNA, 69 bp, Artificial sequence
  Description of the artificial sequence Primer
catatgaaccttagtccaagcagaacaccgatttgcgcggcgctggctgcggccttgctcggagcagct

SEQ ID NO: 4 - DNA, 62 bp, Artificial sequence
  Description of the artificial sequence Primer
ttcgccgcagccgtttctttcgcatgggccggggccagtgcagctgctccgagcaaggccgc

SEQ ID NO: 5 - DNA, 3288 bp, Artificial sequence
  Description of the artificial sequence pHIP-Vector
gaattcgcccttggggatcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgcatgtactaaggaggttgtatggaacaacgcataaccctgaaagattatgcaatgcgctttgggcaaaccaagacagctaaagatcaagaatgttgatcttcagtgtttcgcctgtctgttttgcaccggaatttttgagttctgcctcgagtaatttaccaacactactacgtttaaactgaaacaaactggagactcatatggcgcgccggatccgtcgactcgagttcgacctcgaaagcaagctgataaaccgatacaattaaaggctccttttggagcctttttttttggagattttcaacgtgaaaaaattattattcgcaattcctttagttgttcctttctattctcaccccaagggcgaattccagcacactggcggccgttactagtggatcaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcctagatgcatgctcgagcggccgccagtgtgatggatatctgca

(19) United States
(12) Patent Application Publication
(10) Pub. No.: US 2009/0259035 A1
(43) Pub. Date: Oct. 15, 2009

(54) Method for producing recombinant RNase A

(76) Inventors: Frank Gindullis, Klein Offenseth-Sparrieshoop (DE); Bert Behnke, Ahrensburg (DE); Alexander Caliebe, Altenholz (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/593,663

(22) PCT Filed: Mar. 22, 2005
(86) PCT No.: PCT/EP05/03063

§ 371 Date: Sep. 30, 2008

(30) Foreign Application Priority Data
Mar. 22, 2004 (DE) 10 2004 013 955.5

(51) Int. Cl. C07H 1/06; C12P 21/02; C12N 1/21; C07H 21/02; C12N 9/22

(52) U.S. Cl. 536/124; 435/69.1; 435/252.33; 536/23.1; 435/199

(57) ABSTRACT

The invention relates to a method for producing recombinant RNase A in E. coli. Said method is characterised in that it uses a DNA sequence, which codes for an RNase A of bovine origin and which is adapted to the codon usage in E. coli. The invention relates to nucleic acid molecules, which contain a nucleic acid sequence that has been adapted to the codon usage in E. coli and to recombinant nucleic acid molecules, which contain an nucleic acid molecule of this type and permit the expression of the recombinant RNase A in E. coli.

The present invention relates to a method for producing recombinant RNase A in E. coli, which is characterized in that a DNA sequence is used, which codes for a RNase A of bovine origin and which has been adapted to the codon usage in E. coli. The present invention further relates to nucleic acid molecules containing a nucleic acid sequence, which has been adapted to the codon usage in E. coli, as well as to recombinant nucleic acid molecules containing one of said nucleic acid molecules and allowing the expression of the recombinant RNase A in E. coli.

RNase A is an endoribonuclease, which hydrolyses RNA strands at internal phosphodiester bridges. It is specific for single-stranded RNA and cleaves bonds 3′ from pyrimidines. Thus, pyrimidine 3′-phosphates and oligonucleotides having terminal pyrimidine 3′-phosphates are formed after cleavage with RNase A. RNase A consists of a chain of 124 amino acids, which is intramolecularly linked by four disulfide bridges. RNase A is enzymatically active even in the absence of co-factors and bivalent cations. It is inhibited by heavy metal atoms and by DNA in a competitive manner.

RNase A is employed in various molecular-biological techniques. For instance, when isolating either plasmid DNA from bacterial cells or genomic DNA from eukaryotic cells, RNA is also purified beside DNA, which, in large quantities, leads to increased viscosity of the sample and to a decrease in yield. Thus, the RNA has to be degraded by adding RNase A to enhance quality and quantity of the sample. In a similar way, this also applies to the preparation of recombinant proteins.

Another use of RNase A is in detecting single base mutations in RNA or DNA. In this case, RNase A cleaves at mismatches, for example in RNA-RNA heteroduplexes, which have been formed between a reference wild type RNA and a possibly mutated RNA. The size of the cleaved strand can subsequently be estimated by means of gel electrophoresis.

Finally, RNase A is also employed in RNase protection assays, by which the expression of various genes can be examined simultaneously. Said method is based on the hybridization of sample RNAs to complementary, radioactively labeled RNA probes (ribo samples) and the subsequent digestion of non-hybridized sequences with one or more single-strand-specific ribonucleases. After completion of digestion, the ribonucleases are inactivated and the protected fragments of the radioactively labeled RNA are analyzed by means of polyacrylamide gel electrophoresis and autoradiography.

The multiplicity of molecular-biological applications for RNase A requires isolation of large quantities of the enzyme in high purity. The classical method of producing RNase A comprises its isolation from bovine pancreas. The BSE problems of the past years, however, have resulted in that animal material, in particular material originating from cattle, is not accepted anymore by the authorities in pharmaceutical production for reasons of biological safety. Thus, the use of RNases was entirely omitted during the past years and RNA was rather separated in many biotechnological-pharmaceutical methods by means of alternative, in most cases very costly, methods like chromatography instead.

Thus, there is a need for a method allowing the production of large quantities of RNase A without the necessity of using animal material. This can, in particular, be achieved by means of recombinant production of RNase A.

Recombinant production of RNase A is complicated by four factors, however: (1) RNase A is instable when expressed alone in E. coli; (2) four disulfide bridges have to be formed correctly in order to reconstitute RNase A to form an active protein; (3) expression of RNase A within a cell is possibly cytotoxic and (4) RNase A possibly degrades its very own transcript, which leads to a decrease in expression performance.

In the past, various methods for recombinant expression of RNase A have been tried, which were supposed to overcome these obstacles, but all of which resulted in a rather low yield of RNase A.

In one approach, RNase A was expressed under the control of a heat-inducible promoter. This led to the formation of inclusion bodies and to a yield of about 2 mg/l (McGeehan and Brenner (1989) FEBS Letters 247 (1): 55-56).

The expression of a fusion protein of RNase A with a gene 10 protein from the bacteriophage T7 under the control of an IPTG-inducible promoter also led to the formation of inclusion bodies. After enzymatic cleavage of the fusion protein by means of the protease factor Xa and purification, a yield of 4 to 8 mg/l protein was obtained (Laity et al. (1993) Proc. Natl. Acad. Sci. USA 90: 615-619).

Also a fusion protein consisting of β-galactosidase and RNase A was expressed under the control of the IPTG-inducible β-galactosidase promoter in E. coli. Employing said strategy, a yield of 0.2 mg/l was obtained after purification (Nambiar et al. (1987) Eur. J. Biochem. 163: 67-71).

Likewise, RNase A was expressed under the control of an IPTG-inducible promoter together with a signal peptide, which causes the efficient translocation of the RNase A into the periplasm. The RNase A was released from the periplasm by means of spheroplast/osmotic shock and was purified. Employing said strategy, a yield of 0.1 mg/l was obtained (Tarragona-Fiol et al. (1992) Gene 118: 239-245).

Finally, a combination of a heat-inducible promoter and a signal peptide, which directs the transport of RNase A into the periplasm, was also tested in E. coli cells. Also herein, the periplasmic proteins were released by means of spheroplast/osmotic shock and were purified. Employing said method, a yield of 45 to 50 mg/l could be obtained (Okorokov et al. (1995) Protein Expression and Purification 6: 472-480).

Host cells other than E. coli, like for example Bacillus subtilis and Pichia pastoris, were also used for expressing RNase A. Likewise, with said host cells, yields in the range of only 1 to 5 mg/l could be achieved (Vasantha and Filpula (1989) Gene 76: 53-60; Chatani et al. (2000) Biosci. Biotechnol. Biochem. 64(11): 2437-2444).

Despite the numerous attempts to optimize recombinant RNase A expression, there is thus a need for a method, which allows the production of recombinant RNase A in E. coli at a yield higher than is currently possible in the art.

It is therefore a problem underlying the present invention to provide a method, by means of which recombinant RNase A can be produced in large quantities in E. coli cells.

According to the present invention, this and further problems are solved by means of the features of the main claim.

Advantageous embodiments are defined in the subclaims.

According to the present invention, a method is provided for producing recombinant RNase A in E. coli, characterized in that a DNA sequence is used, which codes for an RNase A of bovine origin and which has been adapted to the codon usage in E. coli.

The genetic code is redundant, as 20 amino acids are specified by 61 triplet codons. Thus, most of the 20 proteinogenic amino acids are coded by several base triplets (codons). The synonymous codons which specify an individual amino acid are not used with the same frequency in a specific organism, however, but there are preferred codons, which are used frequently, and codons which are used more infrequently. Said differences in codon usage are put down to selective evolutionary pressures, and, in particular, to the efficiency of translation. One reason for the lower translation efficiency of rarely occurring codons could be that the corresponding aminoacyl-tRNA pools are depleted and are therefore no longer available for protein synthesis.

Furthermore, different organisms prefer different codons. Thus, for example, the expression of a recombinant DNA originating from a mammalian cell often proceeds only suboptimally in E. coli cells. Therefore, the replacement of infrequently used codons by frequently used codons can enhance expression in some cases.

For many organisms, the DNA sequence of a larger number of genes of which is known, there are tables, from which the frequency of the usage of specific codons in the respective organism can be taken. With the aid of said tables, protein sequences can be relatively exactly back-translated to form a DNA sequence, which contains the codons preferred in the respective organism for the different amino acids of the protein. Tables for codon usage can, inter alia, be found at the following internet addresses: http://www.kazusa.or.jp/Kodon/E.html; http://www.hgmp.mrc.ac.uk/Software/EMBOSS/Apps/cai.html; http://www.hgmp.mrc.ac.uk/Software/EMBOSS/Apps/chips.html; or http://www.entelechon.com/eng/cutanalvsis.html. There are programs available also for reverse translation of a protein sequence, for example the protein sequence of RNase A, to form a degenerate DNA sequence, like for instance at http://www.entelechon.com/eng/backtranslation.html; or http://www.hgmp.mrc.ac.uk/Software.EMBOSS/Apps/backtranseg.html.

In the method according to the present invention, the DNA sequence used for expressing recombinant RNase A has been adapted to the codon usage of the E. coli strain K12.

Adapting the sequences to the codon usage in a specific organism can be performed with the aid of various criteria. On the one hand, the codon most frequently occurring in the selected organism can always be used for a specific amino acid; on the other hand, the natural frequency of the different codons in the selected organism can also be considered, so that all codons for a specific amino acid are inserted into the optimized sequence in the genome of the selected organism according to their natural frequency. Herein, the selection of which base triplet is used at which position can be performed in a random manner. Both strategies for optimizing the DNA sequence with respect to the codon usage have proven to be equally suitable for the method according to the present invention.

The DNA sequence coding for bovine RNase A is optimized at least at 30 positions, preferably at least at 40 positions, particularly preferably at least at 50 positions, and most preferably at least at 60 positions in relation to the codon usage in the E. coli strain K12.

Most preferably, the optimized DNA sequences are the DNA sequences given in SEQ ID NO: 1 and SEQ ID NO: 2, respectively, or DNA sequences, which are identical to the DNA sequences given in SEQ ID NO: 1 and SEQ ID NO: 2, respectively, by at least 90%, preferably by at least 92% or 94%, particularly preferably by at least 96% or 98%, and most preferably by at least 99% over the entire coding sequence.

Sequence identity is determined via a number of programs based on different algorithms. Herein, the algorithms of Needleman and Wunsch or Smith and Waterman achieve particularly reliable results. For sequence comparisons, the program PileUp (Feng and Doolittle (1987) J. Mol. Evolution 25: 351-360; Higgins et al. (1989) CABIOS 5: 151-153) or the programs Gap and Best Fit (Needleman and Wunsch (1970) J. Mol. Biol. 48: 443-453 and Smith and Waterman (1981) Adv. Appl. Math. 2: 482-489) were used, which are contained in the GCG software package (Genetics Computer Group, 575 Science Drive, Madison, Wis., USA).

The sequence identity values given in percent in the above were determined with the program GAP over the entire sequence region with the following settings: Gap Weight: 50, Length Weight: 3, Average Match: 10,000, and Average Mismatch: 0.000.

Unless specified otherwise, said settings were used as standard settings for sequence comparisons.

Without intending to be bound by a hypothesis, it is assumed that the codon-optimized DNA sequences allow a more efficient translation and the mRNAs formed thereof possibly have a longer half-life period in the cell and are therefore more frequently available for translation.

While an “RNase A of bovine origin” has essentially the same amino acid sequence as the native protein occurring in bovine cells, it is not coded by the DNA occurring in bovine cells, however.

In a preferred embodiment, the RNase A is expressed in fusion with a signal peptide, which directs the transport into the periplasmic space. Localization in the periplasmic space prevents possible cytotoxic effects, which could occur due to the expression of the RNase A. Examples for such signal peptides include stII and phoA (Denefle et al. (1989) Gene 85: 499-510), OmpF and LamB (Hoffman and Wright (1985) Proc. Natl. Acad. Sci. USA 82: 5107-5111), PelB (Lei et al. (1987) J. Bacteriol. 169 (9): 4379-4383), OmpT (Johnson et al. (1996) Protein Expression Purif. 7: 104-113), beta-lactamase (Kadonaga et al. (1984) J. Biol. Chem. 259: 2149-2154), enterotoxins LT-A, LT-B (Morioka-Fujimoto et al. (1991) J. Biol. Chem. 266: 1728-1732), and protein A from S. aureus (Abrahmsen et al. (1986) Nucleic Acids Res. 14: 7487-7500).

Various non-natural, synthetic signal sequences, which allow the secretion of specific proteins, are also well known to the person skilled in the art.

The phoA signal peptide is preferably used.

Expression of RNase A is preferably controlled by an inducible promoter. The use of a heat-inducible promoter, wherein the expression of the gene being under its control is induced by an increase in the cultivation temperature to 42° C., is particularly preferred.

A recombinant nucleic acid molecule comprising the following components in an order from 5′ to 3′ is also an object of the present invention:
a promoter active in E. coli,
optionally, a sequence coding for a signal peptide,
a DNA sequence adapted to the codon usage in E. coli, which codes for an RNase A of bovine origin.

Preferably, the promoter is an inducible promoter, particularly preferably a heat-inducible promoter. The signal peptide preferably is a signal sequence, which directs the transport of the protein into the periplasmic space, and most preferably is the phoA signal peptide.

The methods for producing a nucleic acid molecule, which comprises the components listed in the above, are standard molecular-biological methods and can be taken from the literature, like for example Sambrook and Russell (2001) Molecular Cloning—A laboratory manual, 3^{rd} edition, Cold Spring Harbor Laboratory Press, Cold Spring Harbor, N.Y., USA.

Inducing the promoter preferably takes place from the middle toward the end of the exponential growth phase. At this point of time, the cells have reached a wet biomass of about 35 to 50 g/l culture medium. The induction of the protein expression is carried out for at least 14, normally for 14 to 20 hours, preferably for at least 16, normally for 16 to 18 hours, and most preferably for about 17 hours.

Various E. coli strains, inter alia BL21, BNN93, MM294, ATCC 23226, and ATCC 23851, are suitable as host cells for the expression of the recombinant RNase A.

Methods, by means of which the DNA for recombinant expression of RNase A can entry into the host cells, are known to the person skilled in the art. Among those methods are both chemical methods and physical methods like electroporation. Culture media and cultivation conditions suitable for E. coli cells are also known to the person skilled in the art. These can also be taken from the literature, like for example Sambrook and Russell (2001) Molecular Cloning—A laboratory manual, 3^{rd} edition, Cold Spring Harbor Laboratory Press, Cold Spring Harbor, N.Y., USA.

At the time of harvest, subsequent to cultivation and, optionally, induction, the E. coli cell culture in a suitable culture medium contains at least 0.2 g RNase A per litre culture medium, preferably at least 0.5 g/l, particularly preferably at least 1 g/l, and most preferably about 1.2 g RNase A per litre culture medium.

In a preferred embodiment, RNase A forms inclusion bodies in the host cells. These inclusion bodies are insoluble intracellular aggregates of the expressed protein. They can be isolated by centrifugation at low speed and normally consist of almost pure deposits of denatured forms of the recombinantly expressed protein. In the case of RNase A, the formation of inclusion bodies possibly contributes to the high yield, as the product is present in an inactive form and can therefore not exhibit any cytotoxic effects.

In order to release the inclusion bodies, the host cell has to be lysed. Lysis of the cell can be achieved, for example, by means of mechanical shearing stress, enzymatic digestion, for example with lysozyme, ultrasonic treatment, homogenization, glass bead vortexing, treatment with detergents or organic solvents, by means of deep-freezing and thawing or by means of treatment with a denaturing agent (Bollag et al. (1996) Protein Methods, 415 pages, Wiley-Liss, NY, N.Y.). Optionally, the cells can be lysed in the presence of a denaturing agent or a disulfide-reducing agent. Insoluble or aggregated material can be separated from soluble proteins by means of various methods, for example centrifugation, filtration (including ultrafiltration) or precipitation.

In the next step, the insoluble or aggregated material has to be made soluble or monomeric by means of treating it with a denaturing agent. Suitable denaturing agents include: urea, guanidine, arginine, sodium thiocyanate, pH-extremes (diluted acids or bases), detergents (for example SDS, sarkosyl), salts (chlorides, nitrates, thiocyanates, trichloroacetates), chemical derivatization (sulfitolysis, reaction with citraconic anhydride), solvents (2-amino-2-methyl-1-propanol or other alcohols, DMSO, DMF) or strong anion-exchanger resins, like for example Q-sepharose. Suitable concentrations of urea are 1 to 8 M, preferably 5 to 8 M. Suitable concentrations of guanidine are 1 to 8 M, preferably 4 to 8 M. Particularly preferably, guanidine is used in a concentration of 5 M.

Particularly preferably, the solubilizing buffer additionally contains a redox mixture consisting of an oxidizing agent and a reducing agent in order to enhance the reduction of intra- and intermolecular disulfide bridges. Examples for suitable redox mixtures include cysteine/oxygen, cysteine/cystine, cysteine/cystamine, cysteamine/cystamine, and reduced glutathione/oxidized glutathione. Most preferably, the solubilizing buffer contains reduced and oxidized glutathione. Herein, the reduced glutathione is present in a concentration of 1 to 10 mM, preferably 2 to 5 mM, in the solubilizing buffer. The concentration of the oxidized glutathione is 1/10 to 1/1, preferably 1/10, of the concentration of the reduced glutathione.

The pH value of the solubilizing mixture preferably lies between pH 6 and pH 10, particularly preferably it lies between 7.5 and 9.5, most preferably it is about pH 9.

Subsequently to solubilizing the inclusion bodies, the protein has to be refolded to its active form. To this end, the protein has to assume its native conformation and form its native disulfide bridges. Refolding is achieved by reducing the concentration of the denaturing agent, so that the protein can renature to its soluble, biologically active form. The concentration of the denaturing agent can be reduced by dialysis, dilution, gel filtration, precipitation of the protein or by immobilization to a resin, followed by washing with a buffer. Preferably, the concentration of the denaturing agent in a native buffer is reduced by means of dilution.

In order to restore the native disulfide bridges of the protein, an oxidant or a redox mixture consisting of an oxidant and a reducing agent, which catalyze the disulfide exchange reaction, is added. Suitable oxidants include oxygen, cystine, oxidized glutathione, cystamine, and dithioglycolic acid. Examples for suitable redox mixtures include cysteine/oxygen, cysteine/cystine, cysteine/cystamine, cysteamine/cystamine, reduced glutathione/oxidized glutathione, sodiumsulfite/sodiumtetrathionate etc. Optionally, a reducing agent like DTT or 2-mercaptoethanol can be added to the refolding mixture in order to enhance the disulfide exchange. Optionally, a metal ion like copper can be added to the refolding mixture in order to enhance the oxidation of the protein. Suitable concentrations of metal ions in the refolding mixture are 1 μM to 1 mM.

Preferably, the refolding mixture contains reduced and oxidized glutathione. Herein, the reduced glutathione is present in the refolding mixture in a concentration of 1 to 10 mM, preferably 2 to 5 mM. The concentration of the oxidized glutathione is 1/10 to 1/1, preferably 1/10, of the concentration of the reduced glutathione.

Preferably, the pH value of the refolding mixture lies between pH 6 and pH 10, particularly preferably the pH value lies between 7.5 and 9.5, and most preferably the pH value of the refolding mixture is about pH 9.

Subsequently to solubilizing and refolding the RNase A, it is preferably further purified by means of chromatographic steps. Particularly preferably, the chromatography is a cation exchange chromatography, wherein RNase A, at a specific pH value of the buffer, which should at least be 0.5 to 1.5 pH units below the pI value of the RNase A of 9.45, binds to the matrix of the cation exchange column due to its positive total charge, while most of the contaminating proteins do not bind and can be removed by means of washing.

Suitable cation exchange matrices include carboxymethyl (CM) cellulose, AG 50 W, Bio-Rex 70, carboxymethyl (CM) sephadex, sulfopropyl (SP) sephadex, carboxy-methyl (CM) sepharose CL-6B, and sulfonate (S) sepharose.

Suitable matrices and protocols for conducting the cation exchange chromatography can be taken from the product information of suppliers like Amersham Biosciences (http://www.amershambiosciences.com) or Bio-Rad (http://www.bio-rad.com) by the person skilled in the art.

Buffers suitable for cation exchange chromatography include maleate, malonate, citrate, lactate, acetate, phosphate, HEPES and Bicine buffers. Preferably, the concentration of the buffer lies between 20 mM and 50 mM. For purifying RNase A, the pH value of the buffer should, if possible, not exceed 8.0, preferably not exceed 7.0.

Particularly preferably, 20 mM sodium acetate pH 5.0 or 50 mM Tris HCl pH 6.8 is used for the cation exchange chromatography.

Subsequently to washing, RNase A can be eluted from the column by means of an alteration, which is an increase of the pH value or an increase of the ionic strength in the case of cation exchange chromatography.

Preferably, the elution is effected by means of increasing the ionic strength. If 20 mM sodium acetate pH 5.0 is used as buffer, a mixture of 20 mM sodium acetate pH 5.0 and 350 mM NaCl is used for elution. If 50 mM Tris HCl pH 6.8 is used as buffer, Tris HCl pH 6.8 at a concentration of 250 mM is used for elution.

Further suitable conditions for cation exchange chromatography can be taken from appropriate literature, like for example the manual “Ion Exchange Chromatography-Principles and Methods” by Amersham Biosciences, Freiburg, Germany.

Afterwards, RNase A can be further purified by means of additional chromatographic steps like filtration, precipitation, and diafiltration.

Subsequently to purification, DNases co-purified with RNase A have to be inactivated by means of heat treating the product-containing chromatographic fractions at 95° C. in a water bath. Preferably, the heat treatment is performed for 20 to 35 minutes, particularly preferably for about 20 minutes. Alternatively, the heat treatment can also be performed at 80° C. for a correspondingly longer period.

After purification the RNase A can be analyzed with respect to its quantity and activity. Analysis of the amount of RNase A purified can, on the one hand, be performed qualitatively via an SDS-PAGE analysis, followed by staining with Coomassie Brilliant Blue. For quantifying RNase A, a calorimetric assay, like for example the Bradford assay or a chemical reaction like the Lowry or the Biuret reaction, can be carried out. A non-recombinant, commercially available bovine RNase A with a known combination can be employed as standard for analyses. Furthermore, the concentration of the protein solution can also be calculated via extinction at 278 nm, while taking into consideration the molar extinction coefficient of RNase A.

Subsequently to purification, an RNase A yield of more than 100 mg/l culture medium, preferably of more than 200 mg/l culture medium, particularly preferably of more than 250 mg/l culture medium, and most preferably of about 300 mg/l culture medium is obtained by the method according to the present invention.

Subsequently to purification, an RNase A yield of more than 3 mg/g wet biomass, preferably of more than 4 mg/g wet biomass, particularly preferably of more than 6 mg/g wet biomass, and most preferably of more than 8 mg/g wet biomass is obtained by the method according to the present invention.

The activity of the RNase A can be determined by digestion of defined RNA molecules. RNase A activity is expressed in Kunitz units. One Kunitz unit causes an extinction decrease at 300 nm of 100% in one minute at a temperature of 25° C. and a pH value of 5.0, if total RNA from yeast is used as a substrate. In order to compare the activity of the RNase A purified according to the method of the present invention with commercially available, non-recombinant RNase A preparations, the commercially available RNase A preparations can be used as a standard.

RNase A purified according to the method of the present invention has an activity of at least 40 Kunitz units and preferably of at least 50 Kunitz units. Said activity is comparable to the values for non-recombinant RNase A, which are indicated by the manufacturers.

Alternatively, the activity test can also be conducted with different RNAs at different temperatures and for different time periods. Of course, it is furthermore possible to test the activity of RNase A within the scope of the desired final application, for example in form of plasmid isolation.

The purified RNase A can be stored either as lyophilisate or in liquid form. Suitable as buffers for liquid storage are, for example, Tris-HCl at a pH value from 6.8 to 7.4, a phosphate buffer, or sodium acetate. In addition, the buffers can contain further components like glycerol, triton X-100, sodium chloride, or EDTA. If Tris-HCl is used as buffer, it is employed at a concentration of from 10 mM to 250 mM. Preferably, it is employed at a concentration of 250 mM. RNase A is present in the solution at a concentration of from 1 to 100 mg/ml, preferably 100 mg/ml.

RNase A produced by the method according to the present invention is suitable for all applications, wherein RNase A is conventionally used, viz, for example, for isolating plasmid or genomic DNA and recombinant proteins, for detecting single base mutations, or for the ribonuclease protection assay.

The present invention is illustrated by means of the following Examples, which are not to be understood as limiting.

### EXAMPLES

### 1. Optimizing the DNA Coding for RNase A

For optimizing the codon usage, the protein sequence of the mature RNase A, which can be found under the accession number AAB35594 in the NCBI protein database (http://www.ncbi.nlm.nih.gov/entrez), was first reverse-translated. Thereby, a degenerate DNA sequence was obtained, which was subsequently optimized with the aid of publicly accessible codon usage tables (http://www.kazusa.or.jp/Kodon/E.html) for E. coli K12 cells. The DNA sequence for RNase A was adapted to the codon usage in the E. coli strain K12 by means of always using the codon selectable and most frequently used in said cells for one specific amino acid. During optimization, attention was paid that no additional NdeI or SalI restriction sites were generated, as these would have impeded subsequent cloning into the expression vector. The cDNA sequence thus obtained, which was referred to as mRAopt and is given in SEQ ID NO: 1, was synthesized at Geneart (Regensburg, Germany).

The phoA signal peptide originates from the alkaline phosphatase from Lysobacter enzymogenes (Accession No. Q05205). The sequence of the presumed signal peptide (29 amino acids) was back-translated with an Applet by Entelechon (http://www.entelechon.com/), which is to be found on their homepage, while taking into consideration the codon usage in E. coli K12. Derived from said reverse-translated nucleic acid sequence, two single-stranded DNA oligonucleotides (phoA for, phoA rev) were synthesized and linked to the coding cDNA mRAopt via recombinant PCR.

### 2. Cloning of the Optimized DNA into an Expression Vector and Transformation of the E. coli Cells

In a so-called primary PCR reaction, both the oligonucleotides phoA for (SEQ ID NO: 3) and phoA rev (SEQ ID NO: 4) coding for the signal peptide were hybridized to form a double strand and were amplified under the following conditions:

Reaction setup:
10 μl phoA for (10 pmol/μl)
10 μl phoA rev (10 pmol/μl)
10 μl 10×PCR buffer with MgCl₂ (15 mM) (Expand High Fidelity PCR System, Roche, Mannheim, Germany)
4 μl dNTP mix (2 mM each) (Gibco Life Technologies, Eggenstein, Germany)
1 μl polymerase (3.5 units/μl) (Expand High Fidelity PCR System, Roche, Mannheim, Germany)
65 μl H₂O
   PCR conditions:

| | | | |
|---|---|---|---|
| 1^{st} step: | 1 | min | 95° C. |
| 2^{nd} step: | 1 | min | 95° C. |
| | 30 | sec | 50° C. |
| | 30 | sec | 72° C. |
| | 25 | cycles | |
| 3^{rd} step: | 4 | min | 72° C. |
| 4^{th} step: | | | 4° C. |

The reaction product of the primary PCR reaction was employed together with the plasmid containing the optimized DNA sequence (pmRAopt, provided by Geneart), and a primer allowing amplification from the 3′ end of the RNase A opt cDNA (5′ GTC GAC TAT TAG ACG CTC GCA TC 3′), in a so-called recombinant PCR reaction under the following conditions:

Reaction setup:
10 μl pmRAopt (10 ng/μl)
10 μl RNase opt 3′ primer for amplification (10 pmol/μl)
10 μl primary PCR product
10 μl 10×PCR buffer with MgCl₂ (15 mM) (Expand High Fidelity PCR System, Roche, Mannheim, Germany)
8 μl dNTP mix (2 mM each) (Gibco Life Technologies, Eggenstein, Germany)
1 μl polymerase (3.5 units/μl) (Expand High Fidelity PCR System, Roche, Mannheim, Germany)
   PCR conditions:

| | | | |
|---|---|---|---|
| 1^{st} step: | 1 | min | 95° C. |
| 2^{nd} step: | 1 | min | 95° C. |
| | 30 | sec | 55° C. |
| | 45 | sec | 72° C. |
| | 30 | cycles | |
| 3^{rd} step: | 4 | min | 72° C. |
| 4^{th} step: | | | 4° C. |

In the PCR product obtained by said reaction, the DNA sequence for the signal peptide was located at the 5′ end of the RNase A opt cDNA.

The PCR product was purified via an agarose gel and ligated into the vector pCR2.1-TOPO (Invitrogen, Karlsruhe, Germany). The correct sequence was verified by means of sequencing. The cloned PCR product was subsequently excised from the vector via NdeI/SalI double digestion and was purified via an agarose gel. Simultaneously to this, the vector pHIP-pelB-RNase-opt was digested with NdeI/SalI, whereby the linearized vector pHIP having NdeI and SalI ends was obtained. Said vector was also purified via an agarose gel. The NdeI/SalI-digested phoA-RNase A-opt fragment was ligated into the linearized vector and transformed and amplified in E. coli TOP10F′ cells. From said strain, the plasmid DNA was then isolated and used for the calcium chloride-mediated transformation of the E. coli strain ATCC 23226 (like in Sambrook and Russell, vide supra).

The methods employed herein for cloning the optimized cDNA are well known to the person skilled in the art and can be looked up, for example, in Sambrook and Russell (2001), vide supra.

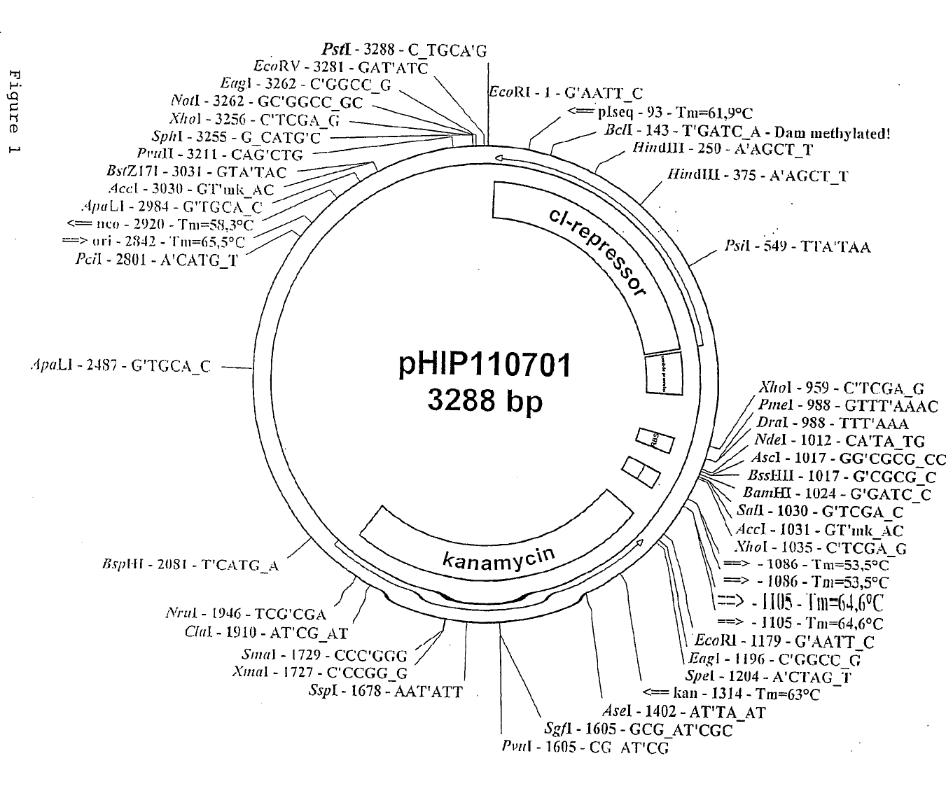

The map of the expression vector pHIP is depicted in FIG. 1, the sequence of the expression vector is given in SEQ ID NO: 5. Said vector already contains the heat-inducible promoter sequence and a multiple cloning site, into which corresponding cDNAs can be cloned via the restriction sites of the enzymes NdeI and SalI.

### 3. Fermentation and Induction of the Bacteria

The bacteria were cultivated in complete medium (soy peptone (27 g/l), yeast extract (14 g/l), NaCl (5 g/l), K₂HPO₄ (6 g/l), KH₂PO₄ (3 g/l), MgSO₄ (0.5 g/l), glycerol (30 g/l)) at 30° C. At specific time intervals, 1 ml culture broth was transferred into a previously weighed Eppendorf reaction tube and the cells were pelleted by centrifugation at 13,000×g for 3 minutes. The supernatant was entirely removed and the tube was weighed once more. From the difference between the weight of the reaction tube with cell pellet [mg] and the weight of the empty reaction tube [mg] divided by the volume of the culture broth the cells were pelleted from, the wet biomass [mg/ml or g/l] can be determined. Determination was conducted in form of a double determination. After a wet biomass of 35 to 50 g/l was reached, the cultivation temperature was increased to 42° C., whereby expression of the recombinant protein was induced. The induction phase took 17 hours.

### 4. Harvest of the Bacteria and Recovery of Native RNase A from Inclusion Bodies

After the induction phase, the cells were harvested by centrifugation and resuspended in 20 mM sodium phosphate buffer pH 7.4, which was used in a quantity of 3.75 ml/g wet biomass. After that the cells were physically disrupted by means of two passages in a high pressure homogenizer (Niro Soavi, Lübeck, Germany) at a pressure of 800±50 bar. The inclusion body fraction was harvested by means of centrifugation (11,000×g for 30 minutes at 4° C.), resuspended in 20 mM sodium phosphate buffer pH 7.4, wherein the buffer was employed in a quantity of 5 g/l inclusion bodies, and pelleted once more by means of centrifugation.

The inclusion body fraction was solubilized in a denaturing redox buffer (5 M guanidine HCl, 50 mM Tris, 1 mM EDTA, 50 mM NaCl, 2 mM glutathione (reduced), 0.2 mM glutathione (oxidized), pH 9, adjusted with NaOH or HCl).

Insoluble components of the inclusion body fraction were removed from the setup by means of centrifugation or filtration. Subsequently, the optical density of the clarified solution was determined at 280 nm (OD₂₈₀ₙₘ).

Refolding the denatured proteins was conducted by dilution in a native dilution buffer (50 mM Tris, 1 mM EDTA, 50 mM NaCl, 2 mM glutathione (reduced), 0.2 mM glutathione (oxidized), adjusted to pH 9 with NaOH or HCl). The volume of the dilution buffer was selected in such a way that an OD₂₈₀ₙₘ of 5 has resulted in the refolding setup after addition of the solubilized inclusion body fraction. This setup was gently stirred for at least 15 hours (150 to 180 revolutions per minute).

### 5. Ion Exchange Chromatography

The refolding setup was clarified by filtration and subsequently concentrated by ultrafiltration and was rebuffered, so that the setup was present in a buffer system (50 mM Tris-HCl pH 6.8) suitable for subsequent chromatography. The setup obtained from ultrafiltration was chromatographically purified via a cation exchanger (SP Sepharose, Amersham Pharmacia, Freiburg, Germany). Unspecific proteins were removed by means of washing with about three column volumes 100 mM Tris-HCl pH 6.8. Elution was conducted in that the salt concentration was increased to 250 mM Tris-HCl pH 6.8.

### 6. Heat Inactivation and Bottling of RNase A

The product-containing chromatographic fractions were identified by measuring the extinction at 280 nm in the chromatographic device, then pooled and heat treated for 20 minutes at 95° C. in a water bath. Emerging precipitate was removed after the heat treatment by means of filtration or centrifugation. The sample was concentrated by ultrafiltration and the RNase A solution was adjusted to a final concentration of 100 mg/ml by adding 250 mM Tris-HCl pH 6.8. The solution was sterilely filtrated and bottled.

### 7. Analysis of Purified RNase A

Recombinant, purified RNase A was qualitatively analyzed by SDS-PAGE with subsequent staining with Coomassie Brilliant Blue, as is described in Sambrook and Russell (2001), vide supra.

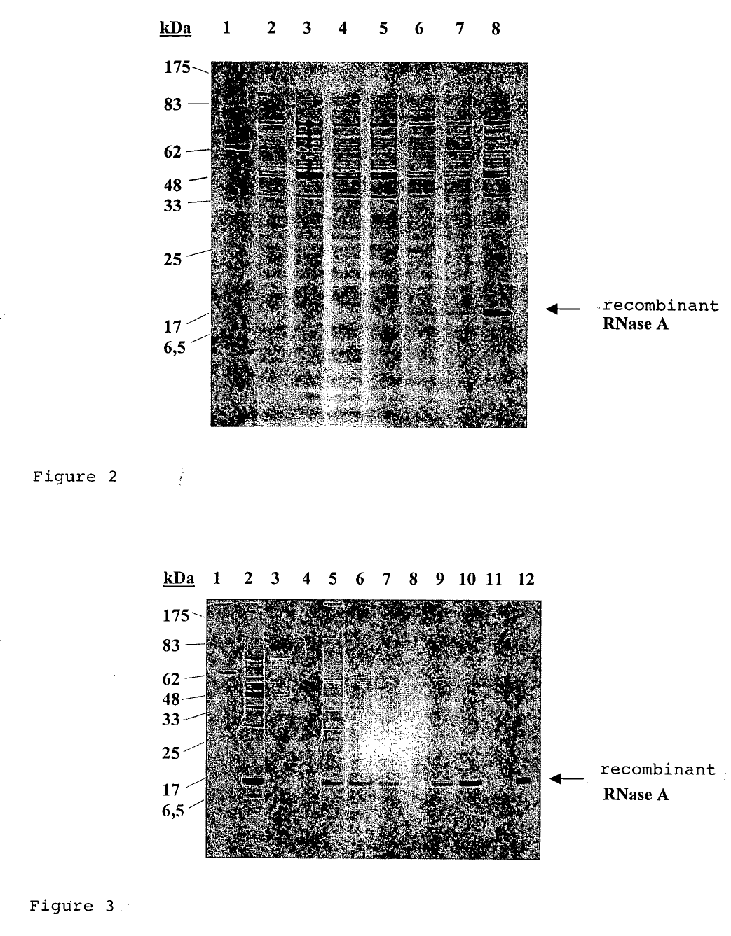

In FIG. 2, the analysis of the protein expression at different points of time during cultivation or induction is depicted. The expression of recombinant RNase A is already detectable 1.5 hours after induction and increases until 18 hours after induction.

FIG. 3 shows the SDS-PAGE analysis of RNase A after the individual purification steps. By means of the sequence of purification steps described in the above, only RNase A is purified.

The concentration of purified RNase A was determined colorimetrically according to Bradford, M. M. ((1976) Anal. Biochem. 72: 248-254).

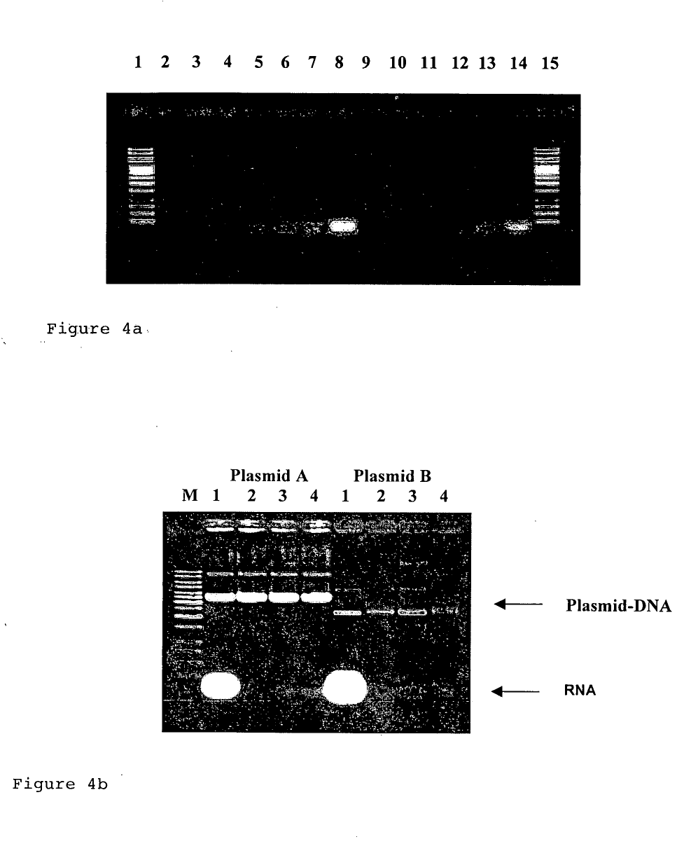

For analyzing RNase A activity, different quantities of RNase A produced by the method according to the present invention were incubated with 10 μl yeast total RNA (10 μg/μl in 100 mM sodium acetate pH 5.0) in a total volume of 20 μl for 5 minutes at room temperature (20° C.). For comparison, a commercially available RNase A was analyzed in parallel. The treated samples were mixed with 4 μl loading buffer, electrophoretically separated on a 1% agarose gel in 1×TAE and made visible by means of staining with ethidium bromide. No significant difference was identifiable between the two RNase A preparations (see FIG. 4a).

Furthermore, both the RNase A produced by the method according to the present invention and two different commercially available RNase A preparations were used for purifying two different plasmids. To this end, the corresponding cultures were incubated over night in dYT medium containing kanamycin (50 μg/ml) at 37° C. and 180 revolutions per minute. 1.5 ml of each setup of each culture were transferred to an Eppendorf reaction tube and pelleted in a table centrifuge for 3 min at 13,000 revolutions per minute. The pellets were each resuspended in 200 μl buffer 1 (Qiagen, Hilden, Germany; 50 mM Tris-HCl (pH 8), 10 mM EDTA, 100 μg/ml RNase A), mixed with 200 μl buffer 2 (Qiagen; 0.2 M NaOH, 1% (w/v) SDS) and incubated for 3 to 5 min at room temperature. Subsequently, 200 μl buffer 3 (Qiagen; 3 M potassium acetate pH 5,5) were added, the samples were mixed by means of inverting and were centrifuged in a table centrifuge for 5 min at 13,000 revolutions per minute. The supernatant (500 μl) was transferred to a fresh reaction tube. 10 μl of each setup were applied onto a 1.2% agarose gel in 1×TAE, electrophoretically separated and then stained with ethidium bromide. Herein, with respect to RNase A activity or DNA conformation, no difference could be observed in the setups with the RNase A according to the present invention and the commercial RNase A preparations (see FIG. 4b).

### FIGURES

1. Map of the pHIP expression vector

The components of the expression vector and the restriction sites of the restriction enzymes are labeled.

2. Examining the expression kinetics of recombinant RNase A in the employed E. coli cells by SDS-PAGE analysis

1: Prestained marker (NEB, Beverly, Mass., USA), 2: 1 h before induction, 3: 0.5 h before induction, 4: induction, 5: 0.5 h after induction, 6: 1.5 h after induction, 7: 2.5 h after induction, 8: 18 h after induction

3. SDS-PAGE analysis of RNase A subsequently to the different purification steps

1: Pre-stained marker (NEB, Beverly, Mass., USA), 2: cell harvest, 3: soluble proteins, 4: supernatant after washing the inclusion body fraction, 5: solubilized inclusion body fraction, 6: after refolding, 7: after diafiltration, 8: flow-through in chromatography (=unbound material), 9: eluate 1, 10: eluate 2, 11: eluate 3, 12: commercial bovine RNase A

4. Activity tests of purified RNase A
a) Digestion of RNA
   1, 15: 1 kb size marker, 2-7; different quantities of commercial bovine RNase A (from 1 μg to 0.025 μg), 8: control without RNase A, 9-14: different quantities of recombinant bovine RNase A (from 1 μg to 0.025 μg)
b) RNase A activity during plasmid isolation
   M: 1 kb size marker, 1: without RNase A, 2: bovine RNase A (Fa. A), 3: bovine RNase A (Fa. B), 4: recombinant RNase A

1. A method for producing recombinant RNase A in E. coli comprising expressing a DNA sequence which codes for a RNase A of bovine origin and which is adapted to the codon usage in E. coli.

2. The method of claim 1, wherein the DNA sequence is adapted to the codon usage of E. coli K12.

3. The method of claim 1, wherein the DNA sequence is adapted to the most frequently used codon in E. coli.

4. The method of claim 1, wherein the DNA sequence corresponds to the DNA sequence given in SEQ ID No. 1, or to a sequence which is at least 90% identical to the DNA sequence given in SEQ ID No. 1.

5. The method of claim 1, wherein the DNA sequence is adapted according to the natural frequency of individual codons.

6. The method of claim 1, wherein the DNA sequence corresponds to the DNA sequence given in SEQ ID No. 2, or to a sequence which is at least 90% identical to the DNA sequence given in SEQ ID No. 2.

7. The method of claim 1, wherein the RNase A is expressed in fusion with a signal peptide, which directs the transport into the periplasmic space.

8. The method of claim 7, wherein the signal peptide is the signal peptide of alkaline phosphatase (phoA).

9. The method of claim 1, wherein the expression of the RNase A is under the control of an inducible promoter.

10. The method of claim 9, wherein the promoter is a heat-inducible promoter.

11. The method of claim 9, wherein induction of the gene expression takes place at the end of the exponential growth phase.

12. The method of claim 9, wherein induction of the gene expression takes place within a period of 14 to 20 hours.

13. The method of claim 1, wherein the RNase A forms inclusion bodies.

14. The method of claim 1, further comprising recovering the RNase A from E. coli cells or the culture medium, optionally by means of solubilisation and refolding of the RNase A.

15. The method of claim 14, wherein the recovering step comprises solubilizing and refolding the RNase A, and guanidine HCl is used as a denaturing agent for solubilisation.

16. The method of claim 14, wherein the recovering step comprises solubilizing and refolding the RNase A, and reduced and oxidised gluthatione is used for refolding.

17. The method of claim 1, further comprising purifying the RNase A chromatographically.

18. The method of claim 17, wherein said chromatographic purifying step is performed by cation exchange chromatography.

19. The method of claim 1, wherein more than 100 mg RNase A per litre culture medium are yielded.

20. The method of claim 1, wherein more than 3 mg RNase A per gram wet biomass are yielded.

21. An E. coli cell culture comprising at least 0.2 g RNase A per litre of culture medium.

22. A nucleic acid molecule comprising the nucleic acid sequence of SEQ ID No. 1.

23. A nucleic acid molecule comprising the nucleic acid sequence of SEQ ID No. 2.

24. A nucleic acid molecule comprising the following components in an order from 5′ to 3′:
a promoter being active in E. coli;
optionally a sequence coding for the signal peptide of claim 7; and
the nucleic acid sequence of SEQ ID No. 1 or 2.

25. A method of producing recombinant RNase A comprising expressing the nucleic acid sequence of SEQ ID No:1 or 2.

26. (canceled)

27. A method of purifying DNA or proteins comprising degrading RNA using the RNase A produced by the method of claim 1.
